(19) United States
(12) Patent Application Publication
(10) Pub. No.: US 2007/0017650 A1
(43) Pub. Date: Jan. 25, 2007

(54) Method and apparatus for providing a solder area on a leadframe

(76) Inventor: Jafry Bin Abdul Wahid, Melaka (MY)

Correspondence Address: BRINKS HOFER GILSON & LIONE, P.O. BOX 10395, CHICAGO, IL, 60610, US

(21) Appl. No.: 11/486,589

(22) Filed: Jul. 14, 2006

Related U.S. Application Data
(63) Continuation of application No. PCT/SG04/00015, filed on Jan. 20, 2005.

(51) Int. Cl. A01H 5/02; B22D 17/24

(52) U.S. Cl. 164/47; 164/81; 164/459; 164/332

(57) ABSTRACT

An apparatus is provided that forms a solder layer on a die pad of a leadframe. Initially, a drop of the solder is deposited on the die pad, and the drop is formed into a layer by the lower surface of a tool which is moved towards the drop. The tool includes a wall which encircles the surface and is resiliently maintained in a position in which the wall encircles and projects downwardly from the surface. Thus, the lower edge of the wall contacts the die pad before the surface forms the solder drop into the layer, and encircles it, thus preventing solder from splashing out.

### RELATED APPLICATIONS

This application is a continuation of PCT Application Serial No. PCT/SG2004/000015, filed Jan. 16, 2004, designating the United States and published in English, which is hereby incorporated by reference.

### BACKGROUND

1. Technical Field

The present invention relates to a method and an apparatus for providing a solder area on a leadframe. In particular, the invention relates to an improved solder dispensing and shaping system.

2. Background Information

Conventionally, integrated circuits (that is “dies”, composed substantially of semiconductor material) are packaged using lead frames. The die is place on a die pad area of the leadframe. The leadframe includes conductive regions (“bond pads”) which are electrically connected to corresponding leads. The bond pads are also connected, normally by wire bonds, to electrical contacts of the integrated circuit. Following the wire bonding operation, a resin body is formed encapsulating the die and the wire bonds. The leadframes may then be cut, so that lead portions of the leadframe project out of the resin. Thus, a package is formed which includes the integrated circuit and leads electrically connected to its corresponding contacts.

It is known to provide an element of solder on the die pad portion of the leadframe to secure the chip to the bond pad and to improve heat transfer from the die to the die pad when the die is in use. FIG. 1 shows the step in a conventional method of this kind. The die pad is indicated as 1, and a drop 3 of liquid solder is applied to it from a dispenser 5. Surface tension in the drop 3 maintains it in a condition in which the top of the drop 3 is significantly spaced from the upper surface of the leadframe 1. The drop 3 is flattened (or otherwise patterned) by a tool 7 having a lower surface 9 (which need not be flat). The tool 7 is gradually lowered, as shown in FIGS. 1(b) to 1(f), to squash the drop 3 so that it is spread out over the lower surface 9. The tool 7 is then removed, leaving the solder as a layer 11, as shown in FIG. 1(g). The tool 7 is conventionally referred to as a “spanker”. FIG. 1(h) shows the die 2 after it has been placed on the layer 11, and when a wire bond 4 is applied to connect to a lead finger 6. Although only one wire bond 4 is shown, typically there are more than one, and often many, extending for example to respective lead fingers. Ideally, the thickness of the solder layer 11 over its whole area should be in the range 30 to 110 microns, and the tilting of the chip (i.e. the degree to which one edge of the chip is higher/lower than the opposite edge) should be less than 60 microns. These parameters help to ensure that the wire bonding step leads to good wire bonds and that, in systems in which the wire bonding locations are determined by a pattern recognition algorithm by viewing the leadframe and the chips on it, that the pattern recognition is reliable.

It may happen that during the operation of FIG. 1 some of the solder is splashed out laterally to either side. Conventionally, this problem is addressed by optimizing the parameters of the system, such as the thickness of the layer 11, the amount of solder originally present in the drop 3, and parameters of the spanker tool 7, such as the speed at which it moves and height of the surface 9 over the upper surface of the leadframe 1. However, the solder splash problem still exists. Furthermore, due to variability in the process, the parameters have to be optimized periodically, such as the beginning of each shift of a new operator.

### SUMMARY OF THE INVENTION

The present disclosure aims to provide a method and apparatus for providing a solder layer on a leadframe.

In general terms, the disclosure proposes that the lower surface of a spanker tool is provided with a wall which surrounds the lower surface, and is resiliently maintained projecting away from it. The lower edge of the wall is initially lower than the level of the spanker surface, so that as the tool approaches the leadframe the wall contacts the die pad before the lower surface of the spanker tool performs the spreading operation.

Preferably, the lower edge of the wall is resiliently maintained at a position such that the edge of the wall is sufficiently spaced from the lower surface of the tool that the wall comes into contact with the leadframe before the spanker surface of the tool contacts the solder drop.

Specifically, an apparatus for providing a solder layer on a portion of a leadframe, the apparatus includes a tool having a surface for contacting an element of the solder provided on the leadframe; a mechanism for moving the tool towards the leadframe and pressing the surface of the tool against the solder element, to thereby form the solder element into the solder layer; and a wall substantially surrounding the surface of the tool and resiliently maintained with the lower edge of the wall projecting away from the surface of the tool, whereby the wall meets the leadframe and substantially encircles the solder element before the tool forms the solder element into the solder layer.

A second aspect includes a method for providing a solder layer on a portion of the leadframe, including dispensing a liquid solder element onto the leadframe; and urging a tool towards the leadframe, the tool having a surface for forming the solder element into the solder layer, and the tool further including a wall resiliently maintained in a position substantially encircling the surface of the tool and with a lower edge projecting away from the surface, wherein the wall meets the leadframe and substantially encircles the solder element before the tool forms the solder element into the solder layer.

Note that the surface of the tool is preferably not flat, but rather profiled to create a desired thickness profile in the solder layer.

Other systems, methods, features and advantages of the invention will be, or will become, apparent to one with skill in the art upon examination of the following figures and detailed description. It is intended that all such additional systems, methods, features and advantages be included within this description, be within the scope of the invention, and be protected by the following claims.

### BRIEF DESCRIPTION OF THE FIGURES

The invention can be better understood with reference to the following drawings and description. The components in the figures are not necessarily to scale, emphasis instead being placed upon illustrating the principles of the invention. Moreover, in the figures, like referenced numerals designate corresponding parts throughout the different views.

FIG. 1, which is composed of FIGS. 1(a) to 1(h), shows schematically a known process for forming a solder layer on the leadframe and mounting a die.

FIG. 2, which is composed of FIGS. 2(a) to 2(g), is a cross-sectional view of a process.

FIG. 3, which is composed of FIGS. 3(a) to 3(e), shows an apparatus for performing the process shown in FIG. 2.

FIG. 4 is an image of a solder layer performing formed by the process of FIG. 2.

FIG. 5 shows an alternative form of the spanker tool of the embodiment of FIG. 3.

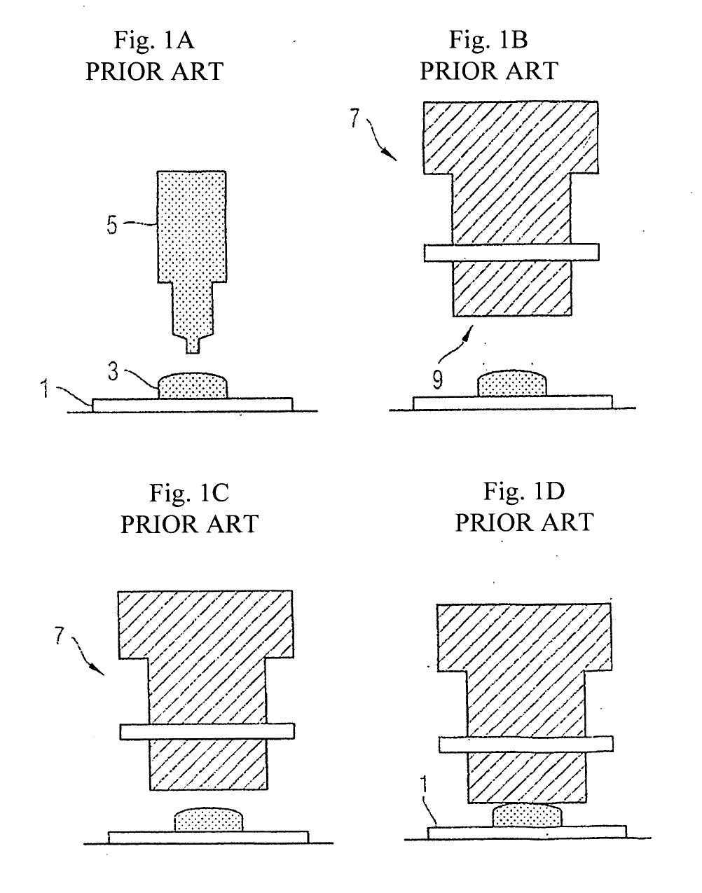

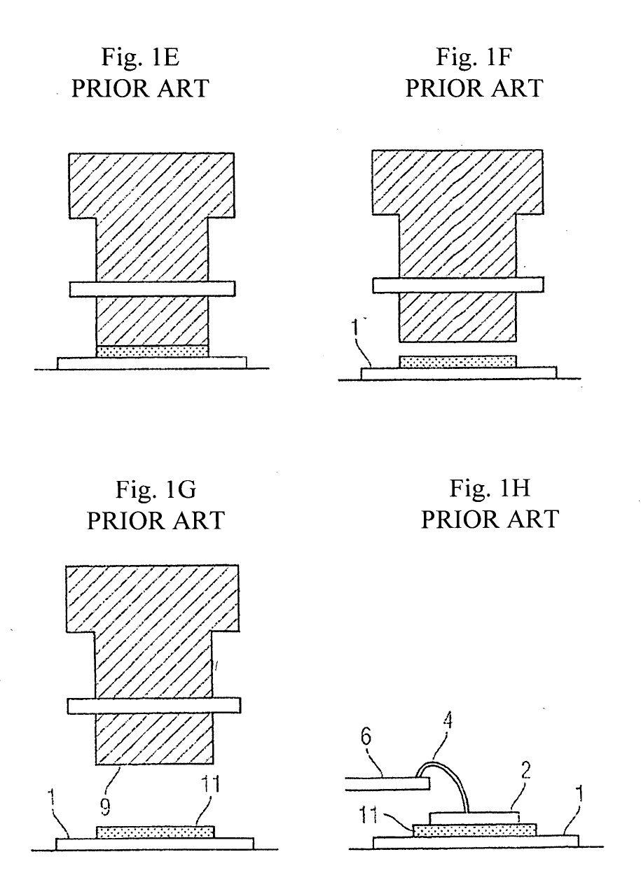

### DETAILED DESCRIPTION OF A PREFERRED EMBODIMENT OF THE INVENTION

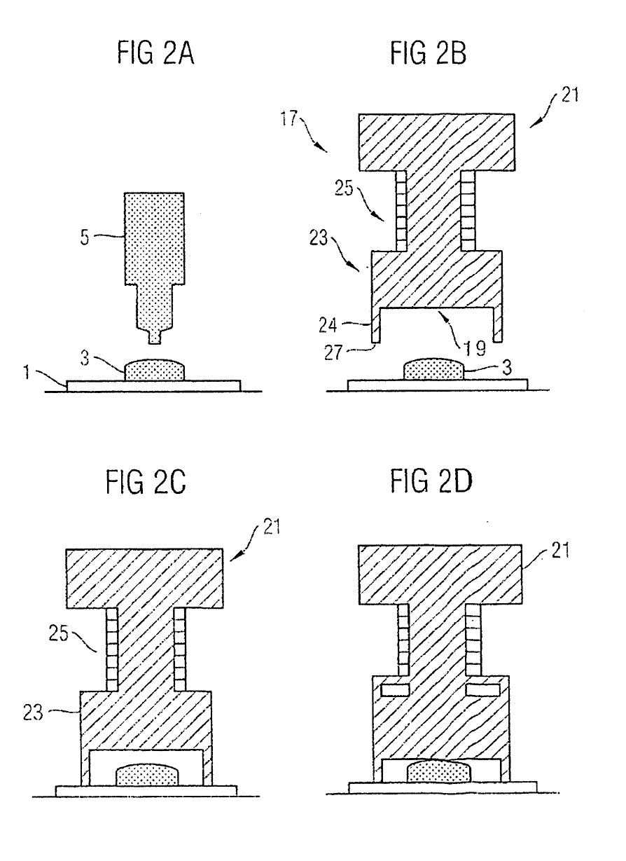

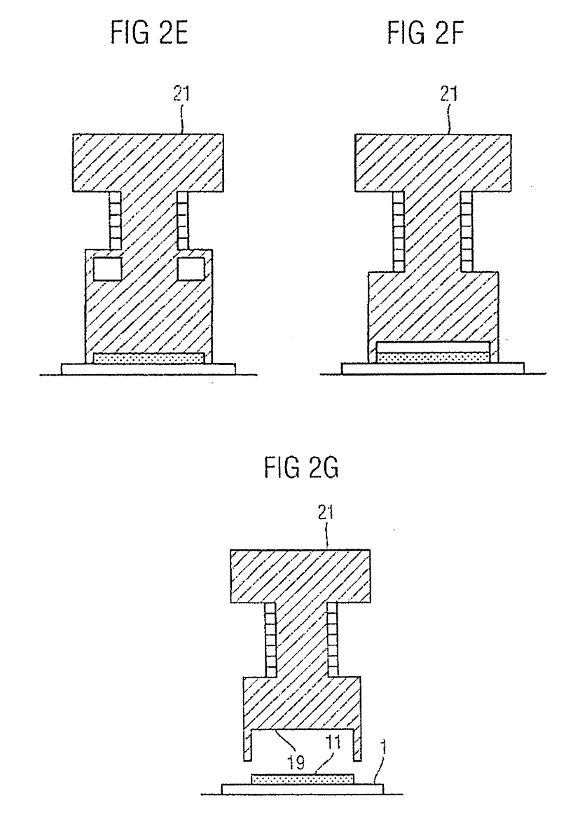

Referring to FIG. 2, the steps of the process are shown. In FIG. 2(a) a drop 3 of liquid solder is deposited onto a die pad portion 1 of a leadframe by a dispenser mechanism 5 which may be in according to known design. Note that the figure is not necessarily to scale. The die pad portion 1 of the leadframe is typically connected to a lead of the leadframe.

As shown in FIG. 2(b) the spanker tool 17 comprises a lower surface 19 and a tool holder 21. It also includes a mechanism (not shown) for moving the tool 17 up and down. The tool 17 further comprises a shroud element 23 which is vertically movable (slidable) relative to the tool 17. The shroud is maintained in a vertical position relative to surface 19 by a spring 25. The spring 25 urges the shroud 23 downwards towards the surface 19, but the tool further includes one or more stop elements for limiting the range of vertical movement of the shroud relative to the surface 19, and in particular the extent to which the spring 25 can force the shroud down. Typically, the spring 25 is one which is able to withstand high temperatures, such as temperatures up to 400° C. The lower portion of the shroud 23 is a wall 24 having a lower edge 27. The lower edge 27 defines a plane which is parallel to the surface of the die pad 1. The wall 24 substantially surrounds the surface 19 (preferably, with no gaps). The wall 24 is preferably not laterally spaced from the edge of the surface 19.

As shown in FIG. 2(c) the tool 17 is moved downward towards the die pad 1 of the leadframe until the lower edge 27 of the shroud of 23 contacts the die pad 1 along a line substantially surrounding the drop 3. At this stage the spring 25 is not compressed.

As shown in FIG. 2(d) as the tool holder 21 continues to move downwardly the leadframe 1 holds the shroud 23 at a fixed vertical position, so that the spring 25 is compressed. In other words, the shroud 23 retracts upwardly relative to the lower surface 19 of the tool 17. Note that the quantity of the solder drop 3 is such that at this time the lower surface 19 of the tool 17 is not contacting it. However, as the tool holder 21 continues to move down, the lower surface 19 of the tool 17 comes in contact with the solder drop 3 and gradually forms it into a layer 11 as shown in FIG. 2(e). The spacing of the lower surface 19 of the tool 17 from the upper surface of the die pad 1 of the leadframe is preferably controlled precisely so as to ensure that the thickness of the layer 11 is as desired. Note that at the time shown in FIGS. 2(d) and 2(e) of the above process, the wall 25 substantially encircles the entire circumference of the solder element 3, and therefore there is no splashing of solder outward at step 2(e). Then, as shown in FIGS. 2(f) and 2(g), the tool holder 21 is gradually moved upwards which removes the surface 19 from the solder layer 11, and subsequently removes the lower edge 27 of the wall 24 from the die pad 1 of the leadframe.

As mentioned above, the upper surface of the solder drop 3 only contacts the lower surface 19 after the lower edge 27 of the shroud 23 has contacted the leadframe 1. This is preferable, because it ensures that the surface 19 applies no force to the drop 3 until the wall 24 is safely in place to limit splashing. However, another possibility would be for the amount by which the lower edge 27 of the wall 24 is spaced downwardly from the surface 19 to be smaller, in comparison with the height of the drop 3, such that the lower surface 19 of the tool 17 contacts the drop 3 at about the same time as the edge 27 of the wall 24 contacts the portion 1 of the leadframe. In this case the wall 24 may still be effective to prevent the splashing of the solder provided that the main force that supplied to the solder by the surface 19 is only applied after the wall 24 contacts the portion 1 of the leadframe.

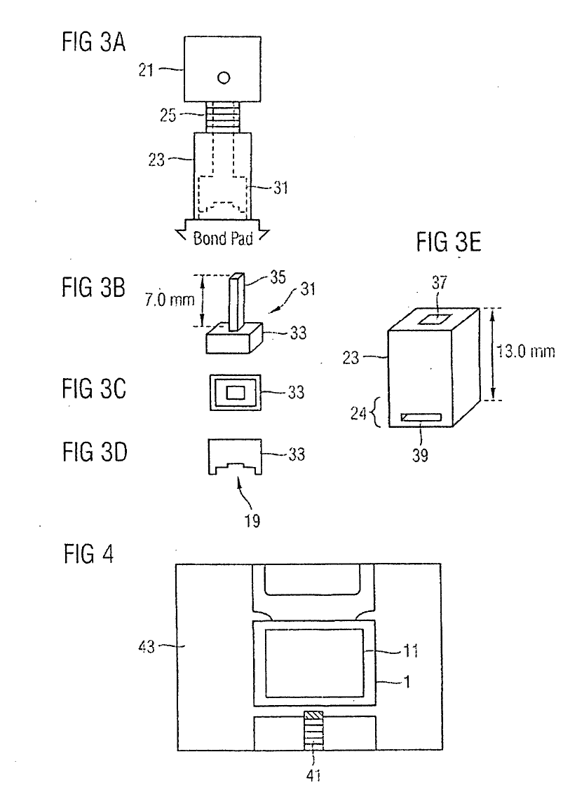

Turning to FIG. 3, a side view is shown of an apparatus which is an embodiment of the invention. Note that the spanker tool 31 having a lower surface 19 is indicated dashed. The spanker tool includes a body portion 33 (having the lower surface 19) and a shaft portion 35, which may for example have the height of 7 mm. The shaft portion 35 is for insertion into an aperture in the tool holder 21. Before this a spring 25 is provided encircling the shaft 35. FIG. 3(c) shows a view of the surface 19 of the body 33, as viewed from the position of the drop 3 (i.e. from underneath). FIG. 3(d) is a cross section of the body 33. As will be seen, the lower surface of the spanker tool 19 is not flat, but includes a number of levels, to effect the shaping of the layer 11. Because the lower surface 19 of the body 33 has a non-uniform profile in the embodiment shown in FIG. 3, the corresponding profile of the layer 11 is also non-uniform, including portions of differing thicknesses. In particular, the center of the lower face of the surface is recessed relative to the outer periphery of the surface 9. The profile of the surface 19 may be selected according to the desired corresponding profile of the layer 11.

FIG. 3(f) is a perspective view of the shroud 23. It has an upper aperture 37 through which the shaft 35 passes in use. Typically the height of the shroud may be about 13 mm. The lower part of the shaft 35 is the wall 24. The shroud also includes an aperture 39 on the side, through which any air trapped during the spanking process is able to escape. Without the aperture 39 there may be a risk in certain embodiments of the invention of solder being sucked up from the bond pad and accumulating at the top of the spanker tool (i.e. in the space between the upwardly facing surface of the body 33 and the downwardly facing surface of the top of the shroud 23.

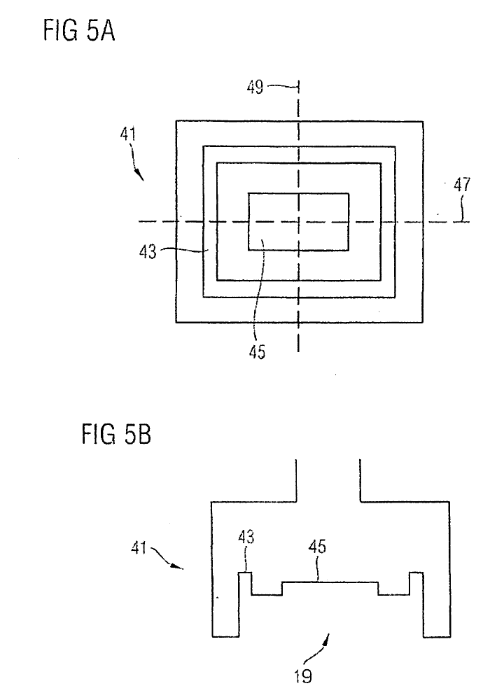

FIG. 5 shows an alternative possible form 41 of the spanker tool 31, from underneath (FIG. 5(a) in a view corresponding to FIG. 3(c)) and in a vertical cross-section (FIG. 5(b), in a view corresponding to FIG. 3(d)). In contrast to the tool 31 of FIG. 3, the spanker tool 41 of FIG. 5 has a different profile of its lower surface 19. In particular, an additional recess 43 is provided in the form of a trench which encircles the recessed center 45 of the lower surface 19 of the spanker tool 41. The lower surface of the surface 19 is mirror-symmetric in the two mirror planes 45, 47 (as is the lower surface of 19 of the spanker tool 31 of FIG. 3 in the two corresponding mirror planes). The recess 43 functions as an air buffer to prevent any excess solder from splashing out. Any such excess solder enters the recess 43, instead of being forced out under the lower edge 27 of the wall 24 of the shroud. In other words, there are two protections from splashing: the wall 24 and the air buffer 43.

Turning to FIG. 4, an image is shown of a layer of solder produced by an embodiment of the invention. As will be seen, no solder has splashed out onto the lead 41 or the heat sink 43.

While various embodiments of the invention have been described, it will be apparent to those of ordinary skill in the art that many more embodiments and implementations are possible within the scope of the invention. Accordingly, the invention is not to be restricted except in light of the attached claims and their equivalents.

1. An apparatus that provides a solder layer on a portion of a leadframe, the apparatus comprising:
a tool having a surface that contacts an element of the solder layer provided on the leadframe;
a wall substantially surrounding the surface of the tool and resiliently maintained with a lower edge of the wall projecting away from the surface of the tool, wherein the wall and the tool are arranged to permit sliding movement relative to each other; and
a mechanism that moves the tool and the wall towards the leadframe and presses the surface of the tool against the solder layer element, to thereby form the solder element into the solder layer;
wherein the wall and the tool are further arranged such that, in use, the wall contacts the leadframe and substantially encircles the solder element at least one of before and at substantially the same time as the tool contacts the solder element.

2. The apparatus of claim 1 further comprising a dispensing mechanism operable to dispense a liquid solder element onto the portion of the leadframe.

3. The apparatus of claim 1 wherein the sliding movement comprises a resilient sliding movement.

4. The apparatus of claim 3 wherein the resilient sliding movement is provided by a spring.

5. The apparatus of claim 4, wherein the spring is located intermediate the tool and the wall and wherein an application of a force to the wall causes the sliding movement.

6. The apparatus of claim 4 wherein the tool comprises a shaft connected to a tool holder, wherein and the spring encircles the shaft.

7. The apparatus of claim 6 wherein the wall and the tool are co-axial, with the wall located about the periphery of the tool.

8. The apparatus of claim 1 wherein the surface of the tool comprises a non-flat profile.

9. The apparatus of claim 8 wherein the surface of the tool includes a central portion that is recessed compared to at least one other area of the surface.

10. The apparatus of claim 8 wherein the surface of the tool includes a recess that receives excess solder.

11. The apparatus of claim 10 wherein the recess comprises a trench that extends around the center of the surface of the tool.

12. A method that provides a solder layer on a portion of a leadframe, the method comprising:
dispensing a liquid solder element onto the leadframe;
urging a tool and a wall towards the leadframe, wherein the tool has a surface parallel to the leadframe, and wherein the wall resiliently maintained in a position substantially encircles the tool;
contacting the wall with the leadframe, the wall substantially encircling the solder element;
contacting the surface with the solder element;
moving the tool in a sliding movement relative to the wall; and
forming the solder element, using the surface, into the solder layer;
wherein contacting the wall with the leadframe occurs before, or at substantially the same time as, contacting the tool with the solder element.

13. The method according to claim 12 wherein forming the solder element further comprises molding the layer to a non-uniform profile corresponding to a non-uniform profile of the surface.

14. The method of claim 12 further comprising draining excess solder while urging the tool towards the leadframe, through a recess in the surface of the tool.

15. The method of claim 14 wherein the recess is a trench which extends around the center of the surface of the tool.

16. The method of claim 12 wherein moving the tool in a sliding movement relative to the wall comprises moving the tool in a resilient sliding movement relative to the wall.

17. An apparatus that provides a solder layer on a portion of a leadframe, the apparatus comprising:
contacting means for contacting an element of the solder layer provided on the leadframe;
a wall substantially surrounding the contacting means and resiliently maintained with the wall projecting away from the contacting means, wherein the wall and the contacting means are arranged to permit sliding movement relative to each other; and
means for moving the contacting means and the wall towards the leadframe and pressing the contacting means against the solder layer element, to thereby form the solder element into the solder layer;
wherein the wall and the contacting means are further arranged such that, in use, the wall contacts the leadframe and substantially encircles the solder element at least one of before and at substantially the same time as the contacting means contacts the solder element.

18. The apparatus of claim 17, further comprising dispensing means for dispensing a liquid solder element onto the portion of the leadframe.

19. The apparatus of claim 18, wherein the contacting means comprises means for receiving excess solder 999

20. The apparatus of claim 19, further comprising resilient means for providing sliding movement, wherein the resilient means is located intermediate the contacting means and the wall and wherein an application of a force to the wall causes the sliding movement.
